(19) United States
(12) Patent Application Publication
(10) Pub. No.: US 2021/0260204 A1
(43) Pub. Date: Aug. 26, 2021

(54) FLUORO-TF-MUC1 GLYCOPEPTIDE CONJUGATE, PREPARATION METHOD AND APPLICATION THEREOF

(71) Applicant: Tianjin University of Science & Technology, Tianjin (CN)

(72) Inventors: Xin MENG, Tianjin (CN); Tingshen LI, Tianjin (CN); Qiang YA, Tianjin (CN); Xujing LIAN, Tianjin (CN)

(21) Appl. No.: 17/007,805

(22) Filed: Aug. 31, 2020

Related U.S. Application Data
(63) Continuation of application No. 16/995,279, filed on Aug. 17, 2020, now abandoned.

(30) Foreign Application Priority Data
Feb. 26, 2020 (CN) 202010118019. 1

(51) Int. Cl. A61K 47/64; C07K 19/00; A61K 39/385

(52) U.S. Cl. CPC A61K 47/646; A61K 39/385; C07K 19/00

(57) ABSTRACT

In a fluoro-TF-MUC1 glycopeptide conjugate, a glycopeptide has a clear and single chemical structure, and can be synthesized in large quantities by chemical methods. The linker selected is easy to be activated, which can be coupled with a carrier protein with high efficiency, and improve the glycoprotein load of the carrier protein. The introduction of fluorine atoms can enhance the stability of glycosidic bonds in glycopeptide antigens, thereby improving the metabolic stability, fat solubility and bioavailability of glycopeptide antigens, and solving the problems of poor immunogenicity and instability of natural tumor-associated MUC1 glycopeptide. Enzyme-linked immunoassay (ELISA) shows that the antibodies in the serum after immunization can recognize the natural MUC1 and achieve cross recognition.

### CROSS REFERENCE TO RELATED APPLICATION

This application claims the priority of Chinese Patent Application No. 202010118019.1 entitled “Fluoro-TF-MUC1 glycopeptide conjugate, preparation method and application thereof” filed with China National Intellectual Property Administration on Feb. 26, 2020, which is incorporated herein by reference in its entirety.

### SEQUENCE LISTING

A Sequence Listing in ASCII text format, submitted pursuant to 37 C.F.R. § 1.821, entitled BGAO8PUS02CON_SEQLISTING.txt, 617 bytes in size, created on Dec. 10, 2020 and filed via EFS-Web, is hereby incorporated by reference in its entirety.

### TECHNICAL FIELD

The present application belongs to the technical field of tumor vaccine development, especially relating to a fluoro-TF-MUC1 glycopeptide conjugate, preparation method and application thereof.

### BACKGROUND ART

The TF antigen is a precursor of Core 2 O-glycan, composed of unsialylated Core 1 structure (Galβ1-3GalNAcαSer/Thr), which was originally discovered by Thomsen and Friedenreich on the glycoproteins that determine blood type in red blood cells. It may be masked by further glycosylation or modification by sialic acid on normal cells. However, TF antigens are exposed on the surface of many cancer cells, including breast cancer, lung cancer, bladder cancer, prostate cancer and pancreatic cancer. Therefore, the expression of TF antigen has been used as a tool for tumor detection and as a standard for recovery, and its distribution density can be used to predict the histopathological grade of cancer, the invasion potential of cancer cells, and the possibility of early recurrence of breast, bladder, and prostate tumors. Georg Springer is a pioneer in the study of TF antigen in breast cancer. He first showed that all breast cancers express TF antigens, and used monoclonal antibody research to prove that TF antigens are only expressed at low level in normal epithelial or breast cells.

Mucin 1 (MUC1) is a type I transmembrane glycoprotein composed of two subunits. (SEQ ID NO: 1) A highly glycosylated extracellular N-terminal domain structure (MUC1-N), protrudes from the surface of the cell up to 200˜500 nm, and is composed of 20 amino acid residues sequence of tandem repeat (HGVTSAPDTRPAPGSTAPPA) with the number of repeats varying from 20 to 120, and there are five potential translated O-glycosylation sites in the sequence located in the serine (Ser) and threonine (Thr) residues. MUC1 belongs to the category of high expression of tumor-associated antigens, and high expression and abnormal glycosylation of MUT-1 are present in tumor cells of many cancers. Studies have shown that nearly 86% of adenocarcinomas have abnormally high expression of MUC-1. The glycosylation pattern on the surface of tumor cells has changed, and is usually covered by tumor-associated carbohydrate antigen (TACA). Thus, MUC1 plays a carcinogenic function through the interaction between TACA and lectin. These interactions often lead to the formation of pre-tumor microenvironment, which is conducive to tumor progression, metastasis and tumor escape. At the same time, due to the shortened glycosylated side chain of MUC1 on the surface of tumor cells, new peptide epitopes appeared, with excessive expression of Tn(GalNAcα-O-Ser/Thr), TF (Galβ1-3 GalNAcα-O-Ser/Thr), sTn NeuAcα2-6-GalNAcα-O-Ser/Thr and other antigens, showing high immunogenicity, so MUC1 has become a potential target for therapeutic tumor vaccines.

Protein conjugates have been widely used to prepare candidate vaccines for a long time. Different protein vectors, such as BSA (bovine serum albumin), KLH (keyhole hemocyanin), TTox (tetanus toxoid) and so on, have been used to conjugate MUC1 glycopeptide to generate an immune response, because these protein carriers contain many epitope antigens and have high immunogenicity.

In addition to coupling the MUC1 glycopeptide to the carrier protein, the immunogenicity of the MUC1 glycopeptide vaccine can be further enhanced by derivation or modification of tumor-associated glycoantigens (TACAs). Since MUC1 glycopeptides belong to endogenous structures and are T cell-independent autoantigens, they are easily tolerated by the immune system, so in order to improve the immunogenicity of these endogenous structures, fluorine atoms can be used in place of the hydroxyl group in sugar molecule, the hydroxyl group is expected to use the isosteric of electrons to enhance the metabolic stability and bioavailability of the glycoantigen, thereby further improving the immunogenicity of the MUC1 glycopeptide antigen.

Through the search, no patent publications related to the present application have been found.

### SUMMARY OF THE INVENTION

The purpose of the present application is to overcome the shortcomings in the prior art, and to provide a fluoro-TF-MUC1 glycopeptide conjugate, preparation method and application thereof. The fluoro-TF-MUC1 glycopeptide conjugate can simulate high titers of IgG antibody levels, so it can be used in vaccines.

The technical scheme adopted by the present application to solve its technical problems are:

a fluoro-TF-MUC1 glycopeptide conjugate, the structural formula of the glycopeptide conjugate is as follows:

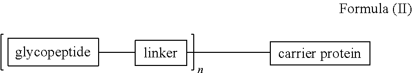

Wherein, the glycopeptides include the glycopeptides in the following general formula (I):

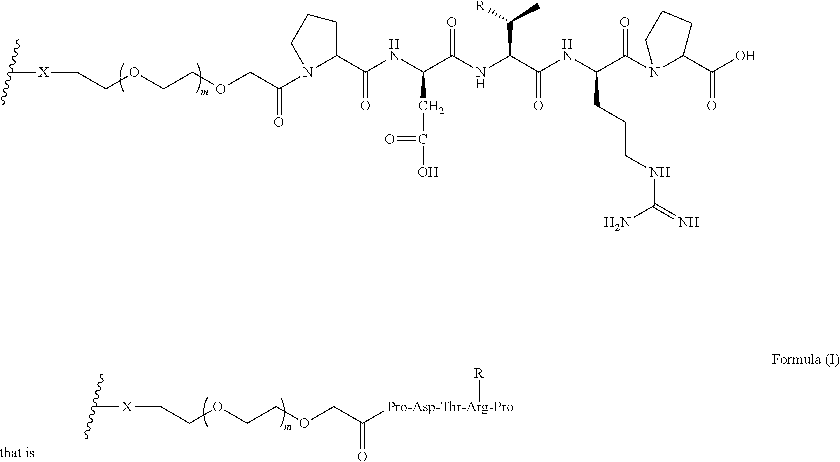

In formula (I) and formula (II):

m includes integers from 0 to 30;

R includes GalNAcα, GalNAcβ, GlcNAcα, GlcNAcβ, Galβ1-3GalNAcα, Galβ1-3GalNAcβ and fluoro derivatives of the glycogroups thereof;

X includes —CH₂, —NH—, —O—, —C(O)—, —S—, and

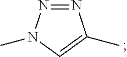

The linker includes a structural part obtained by directly or indirectly connecting the glycopeptide with the carrier protein;

n is the number of oligosaccharides linked to the carrier protein, and n includes integers from 0 to 30;

the carrier proteins include bovine serum albumin, human serum albumin, hemocyanin, tetanus toxin, diphtheria toxin and non-toxic mutant of diphtheria toxin.

Moreover, the general structural formulas of the glycopeptide conjugate include the follows:

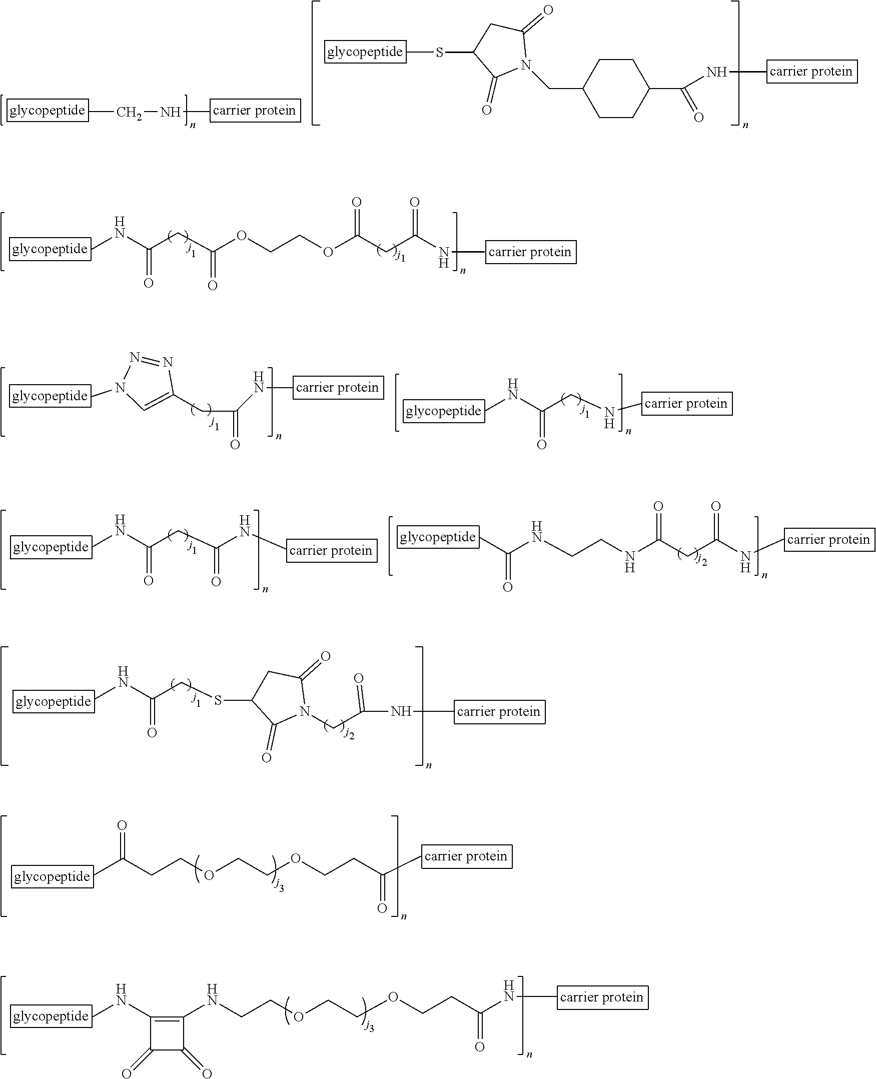

In the formula, j₁ includes integers from 0 to 10, j₂ includes integers from 0 to 10, j₃ includes integers from 0 to 10, and n includes integers from 0 to 30.

Moreover, the general structural formulas of the glycopeptide conjugate include the follows:

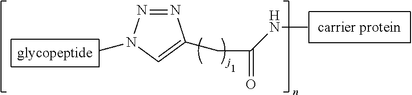

In the formula, j₁ includes integers from 0 to 10, and n includes integers from 0 to 30.

Moreover, the general structural formulas of the glycopeptide conjugate include the follows:

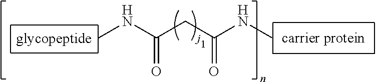

In the formula, j₁ includes integers from 0 to 10, and n includes integers from 0 to 30.

Moreover, the general structural formulas of the glycopeptide conjugate include the follows:

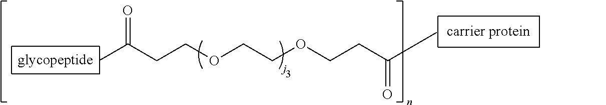

In the formula, j₃ includes integers from 0 to 10, and n includes integers from 0 to 30.

Moreover, the general structural formulas of the glycopeptide conjugate include the follows:

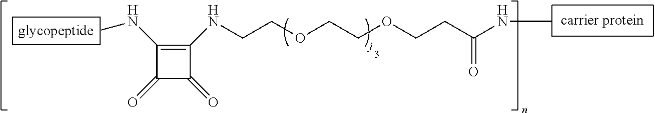

In the formula, j₃ includes integers from 0 to 10, and n includes integers from 0 to 30.

A preparation method of fluoro-TF-MUC1 glycopeptide conjugate as described above includes the following technical route:

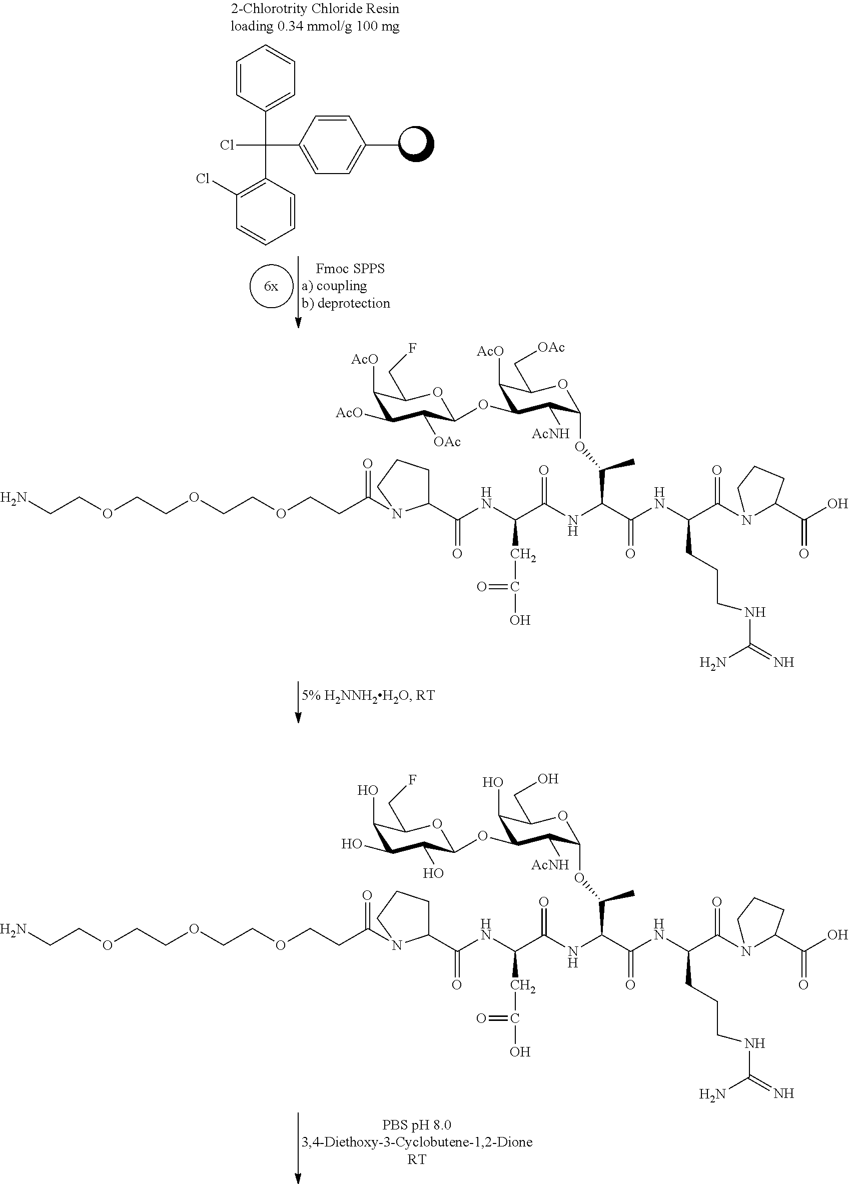

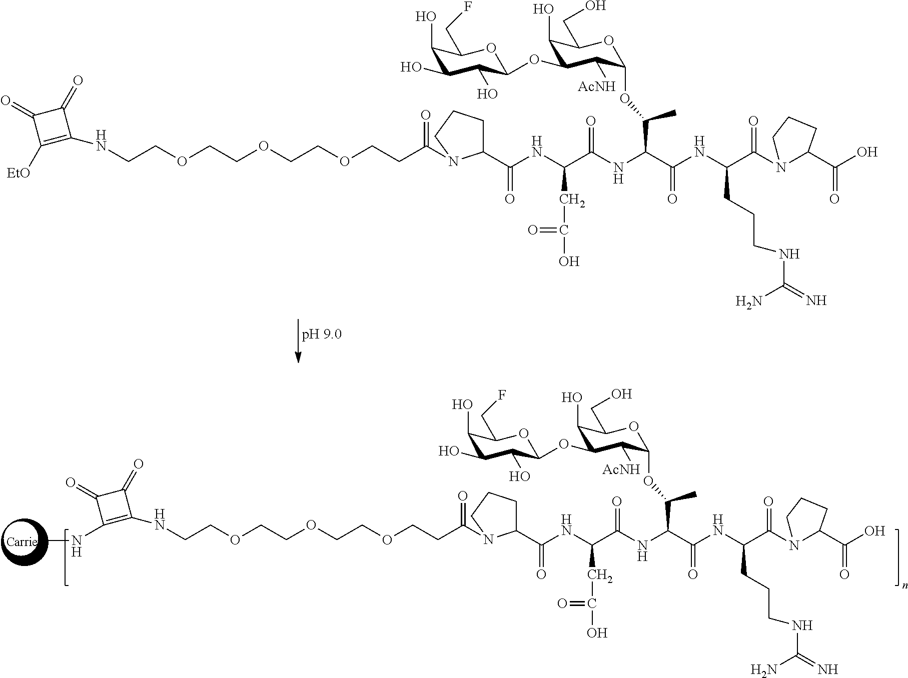

The use of the fluoro-TF-MUC1 glycopeptide conjugate as described above in the preparation of vaccines.

Moreover, the vaccine is a tumor vaccine.

The advantages and positive effects achieved by the present application are:

1. In the fluoro-TF-MUC1 glycopeptide conjugate of the present application, the chemical structure of the glycopeptide is clear and single, and can be synthesized in large quantities by chemical methods.

2. The linkers selected in the present application are easy to be activated, which can efficiently realize the coupling with the carrier proteins and improve the glycopeptide load of the carrier proteins.

3. The introduction of fluorine atoms of the present application can enhance the stability of glycosidic bonds in glycopeptide antigen, thereby improving the metabolic stability, fat solubility and bioavailability of glycopeptide antigens, and thus solving the problems of poor immunogenicity and instability of natural tumor-associated MUC1 glycopeptide.

4. Enzyme-linked immunoassay shows that the antibodies in the serum after immunization with fluoro-MUC1 glycopeptide antigen can recognize natural MUC1 and achieve cross recognition.

5. Animal experiments show that the fluoro-TF-MUC1 glycopeptide conjugate can activate an effective T cell response and produce high titers of IgG antibody levels.

6. As a therapeutic vaccine, the tumor vaccine of the application can reduce the dosage of tumor chemical drugs in the treatment process, reduce the side effects of anti-cancer drugs, and improve the survival rate of cancer patients.

### BRIEF DESCRIPTION OF THE DRAWINGS

FIG. 1 shows the ¹HNMR spectrum of compound 7 of an embodiment;

FIG. 2 is the MALDI-TOF MS spectrum of compound 10 of an embodiment;

FIG. 3 is an analytical HPLC of compound 10 of an embodiment;

FIG. 4 is a MALDI-TOF MS spectrum of the fluoro-TF-MUC1 glycopeptide conjugate of an embodiment;

FIG. 5 is a graph of the titers of specific tri-immune antibody of the fluoro-TF-MUC1 glycopeptide conjugate of an embodiment.

### DETAILED DESCRIPTION OF THE EMBODIMENTS

The embodiments of the present application are described in detail below. It should be noted that the embodiments are only for the descriptive but not restrictive purpose, and cannot limit the scope of protection of the present application.

The raw materials used in the present application, unless otherwise specified, are all conventional commercial products; the methods used in the present application, unless otherwise specified, are all conventional methods in the art.

A fluoro-TF-MUC1 glycopeptide conjugate, the structural formula of the glycopeptide conjugate is as follows:

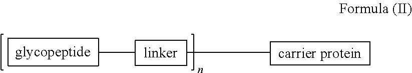

wherein, the glycopeptides include the glycopeptides in the following general formula (I):

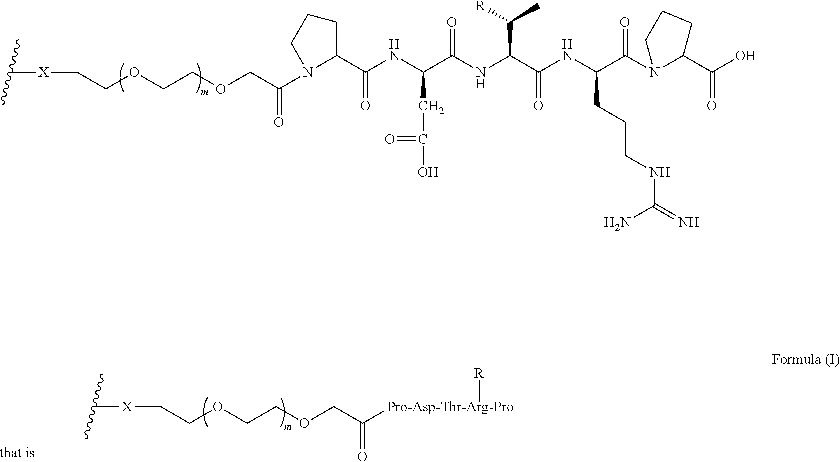

In formula (I) and formula (II):

m includes integers from 0 to 30;

R includes GalNAcα, GalNAcβ, GlcNAcα, GlcNAcβ, Galβ1-3 GalNAcα, Gal 1-3 GalNAcβ and fluoro derivatives of the glycogroups thereof;

X includes —CH₂, —NH—, —O—, —C(O)—, —S—, and

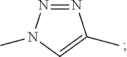

The linker includes a structural part obtained by directly or indirectly connecting the glycopeptide with the carrier protein;

n is the number of oligosaccharides linked to the carrier protein, and n includes integers from 0 to 30;

the carrier proteins include bovine serum albumin (BSA), human serum albumin (HSA), hemocyanin (KLH), tetanus toxin (TT), diphtheria toxin (DT) and non-toxic mutant of diphtheria toxin (CRM197).

In some embodiments, the general structural formulas of the glycopeptide conjugate include the follows:

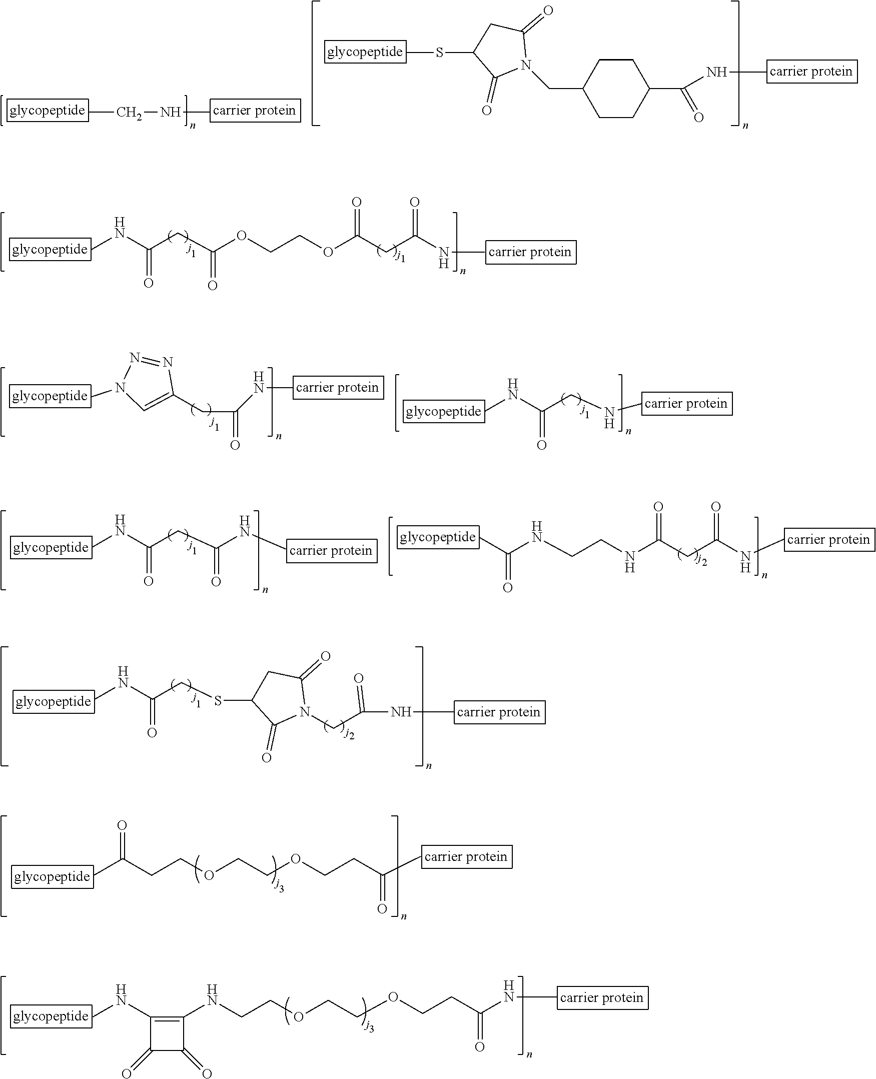

In the formula, j₁ includes integers from 0 to 10, j₂ includes integers from 0 to 10, j₃ includes integers from 0 to 10, and n includes integers from 0 to 30.

In some embodiments, the general structural formulas of the glycopeptide conjugate include the follows:

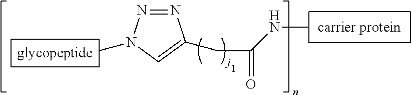

In the formula, j₁ includes integers from 0 to 10, and n includes integers from 0 to 30.

In some embodiments, the general structural formulas of the glycopeptide conjugate include the follows:

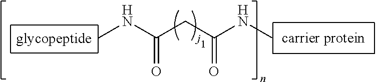

In the formula, j₁ includes integers from 0 to 10, and n includes integers from 0 to 30.

In some embodiments, the general structural formulas of the glycopeptide conjugate include the follows:

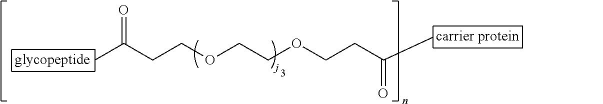

In the formula, j₃ includes integers from 0 to 10, and n includes integers from 0 to 30.

In some embodiments, the general structural formulas of the glycopeptide conjugate include the follows:

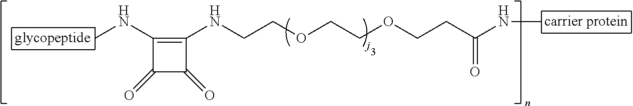

In the formula, j₃ includes integers from 0 to 10, and n includes integers from 0 to 30.

A preparation method of fluoro-TF-MUC1 glycopeptide conjugate as described above includes the following technical route:

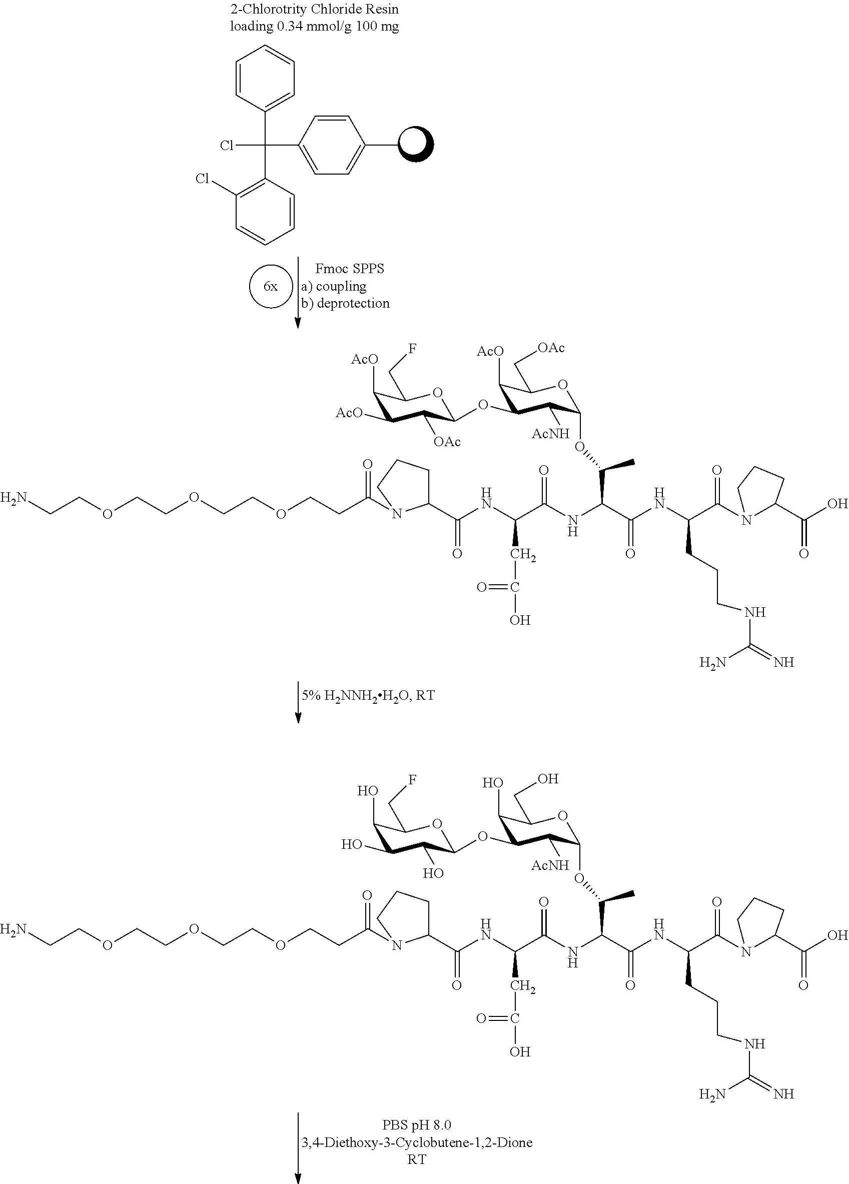

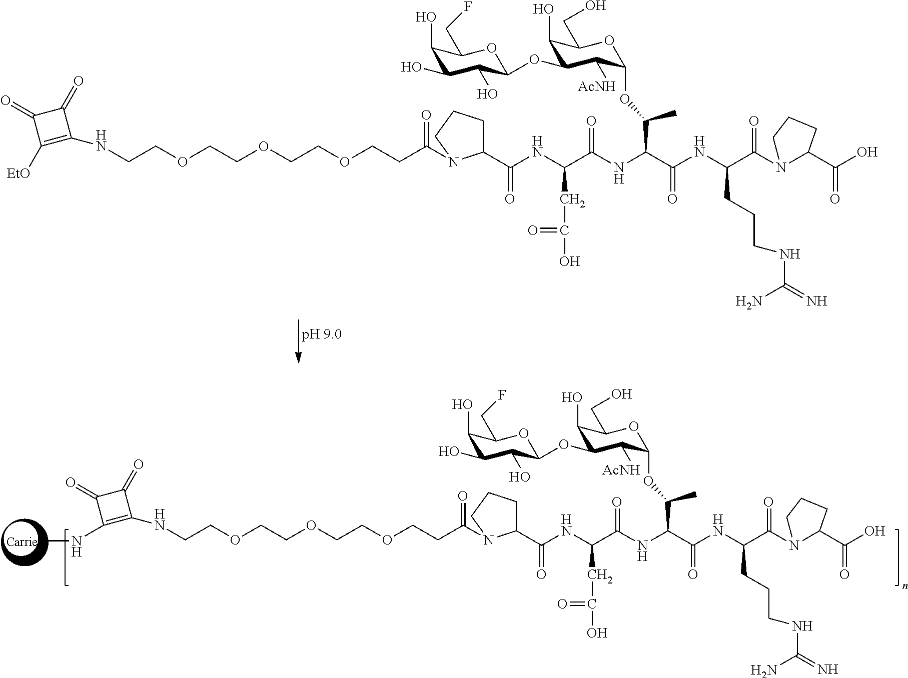

Fluoro-TF-MUC1 glycopeptide fragment is achieved by Fmoc-protected peptide solid-phase synthesis method, that is, by activating the resin, loading amino acids, removing the Fmoc protecting group, repeating the second and third steps as many times as necessary, and finally loading the linker, removing the Fmoc protecting group, cracking, and finally obtaining the glycopeptide containing the protecting group, then removing the protecting group with hydrazine hydrate, activating the glycopeptide with active ester, and in the end, coupling the activated glycopeptide and the protein to prepare fluoro-TF-MUC1 glycopeptide conjugate.

The above preparation method only shows one example of the methods for preparing the compound of formula (II) of the present application. The preparation method of the compound of the present application is not limited to these methods. In the embodiments of the present specification, since the preparation method of the compound of the present application is described in a specific way, therefore, those skilled in the art, according to the above description and the description of the specific embodiments can manufacture fluoro-TF-MUC1 glycopeptide conjugate via appropriate modifications when necessary.

Specifically, the present application provides a general synthesis method for fluoro-TF-MUC1 glycopeptide conjugate, comprising the steps of:

(1) Synthesis Reaction of Carbohydrate Antigen

The glycosy donor (2.0 equivalents) and the sugar amino acid acceptor were dissolved in anhydrous dichloromethane, after stirring at room temperature for 30 min, the system was placed in a low temperature reaction bath at −20° C., trimethylsilyl trifluoromethanesulfonate (0.4 equivalent) was added dropwise to the system and reacted for 1 h. When TLC detection showed that the reaction was complete, triethylamine was added dropwise to quench the reaction. After filtration, vacuum concentration, separation and purification by silica gel column chromatography, intermediate 1 was obtained (the eluent used was petroleum ether (PE)/ethyl acetate (EA)). Followed by a conversion operation of the protective group of the intermediate 1, the intermediate 1 was dissolved in 80% glacial acetic acid solution and reacted at 90° C. for 4 h to remove the benzylidene protective group. Zinc powder (15.0 equivalent) and saturated copper sulfate solution were added to the mixed solution of tetrahydrofuran/acetic anhydride/glacial acetic acid (3:2:1, V/V/V) to reduce the azido group to nitrogen acetyl group, after that, the acetylation protection of exposed hydroxyl group was carried out under the condition of pyridine/acetic anhydride (50.0 equivalences), and finally methanol was used as solvent, 10% palladium carbon was added, hydrogen gas was injected, and the mixture reacted at room temperature for 1 h, after completion of the reaction by TLC monitoring, filtration, vacuum concentration, purification by silica gel column chromatography were conducted, and the target product (the eluent was petroleum ether (PE)/ethyl acetate (EA)) was obtained.

(2) Solid Phase Synthesis Reaction

First, in the resin activation stage, 2-chlorotrityl chloride resin (1.0 equivalent) were put into the solid-phase synthesis tube, dried overnight in vacuum at room temperature, anhydrous DCM was added, shaken at 28° C., 240 r/min for 30 min, and drained; in the second step, the first amino acid was loaded, and the target amino acid (2.0 equivalents) protected by Fmoc was weighed and dissolved in the DCM, and was added into the solid phase synthesis tube after ultrasonic dissolution, and then N, N-Diisopropyl ethylamine (9.0 equivalents) was added. The solid phase synthesis tube was placed in a shaker (28° C., 240 r/min) and shaken for 3 h, methanol was added, and followed by shaking for 30 min and filtration. The resin was washed alternately with DMF/MeOH/DCM, and finally drained and dried in vacuum; the third step was to remove Fmoc: 30% morpholine (200.0 equivalent) was added into the resin and shaken for 30 min, the resin was drained, the operation was repeated once. Then a small amount of resin was taken into a test tube with ninhydrin/phenol chromogenic agent was added dropwise, and the tube was placed in an oil bath at 120° C. The color change of resin was observed after 5 min. The reaction is complete if all the resin turns blue. The resin was washed with DMF/isopropanol/DMF in sequence after the reaction was complete, and drained; in the fourth step, the second amino acid was loaded, and the target amino acid (3.0 equivalents) protected by Fmoc, HBTU (3.0 equivalents) and HOBt (3.0 equivalents) were weighed and dissolved in the DMF, the subsequent operations were the same as the second and third steps above; the fifth step was to load the third amino acid, carbohydrate antigen which was prepared in step (1) (1.5 equivalents), HATU (2.0 equivalents) and HOAt (2.0 equivalents) were weighed and dissolved in the NMP, N-Methylmorpholine (4.0 equivalents) was added, the subsequent operations were the same as the second and third steps above; in the sixth step, the fourth and fifth amino acid were loaded, and the conditions were the same as the fourth step; the seventh step was to load a linker, the linker protected by Fmoc, HATU (3.0 equivalents) and HOAt (3.0 equivalents) were added and dissolved in N-methylpyrrolidone, N-methylmorpholine (6.0 equivalents) were added, and the subsequent operations were the same as the second and the third steps above; the last step, the cracking reaction: the lysate (TFA:TIS:H₂O=15:0.9:0.9) was prepared and added in resin, after shaking (34° C., 240 r/min) for 2 h, filtrated, cooled, the glycopeptide compounds were obtained by recrystallization.

Preparation of Glycopeptide Conjugate

Deprotection of glycopeptide: the glycopeptide compound prepared in step (2) above was put into a small flask, a certain volume of hydrazine hydrate was added. The mixture was stirred at room temperature for 3 h, after that rotary evaporation and concentration, and purification with C18 reverse-phase semi preparative column were conducted to obtain the deprotected glycopeptide compound.

Activation of glycopeptide: the deprotected glycopeptide was dissolved in a mixture of ethanol (EtOH) and water (H₂O) (EtOH:H₂O=1:1), active ester (6.0 equivalents) were added, the pH of reaction solution was adjusted to 8.0 by adding saturated sodium carbonate solution dropwise, and reacted at room temperature for 3 h. After that, it was concentrated, purified with a C18 reverse-phase semi preparative column, and the activated glycopeptides was obtained by lyophilizing.

Synthesis of glycopeptide protein conjugate: the activated glycopeptide and protein (glycopeptide:protein=48:1) were dissolved at a molar ratio of 48:1 in a buffer solution (0.07 M Na₂B₄O₇/0.035 m NaHCO₃, pH 9.0), the mixture was placed in a shaker and shaken slowly at room temperature for 2 days. After that, the glycopeptide protein conjugate was obtained by ultrafiltration and lyophilizing.

More specifically, the relevant preparation and detection are as follows:

(a) Synthesis of N-fluorene methoxycarbonyl-O-(2,3,4-tri-O-acetyl-6-deoxy-6F-β-D-galactopyranosyl-(1→3)-2-acetylamino-2-deoxy-4,6-di-O-acetyl-α-D-galactopyr anosyl)-L-threonine7

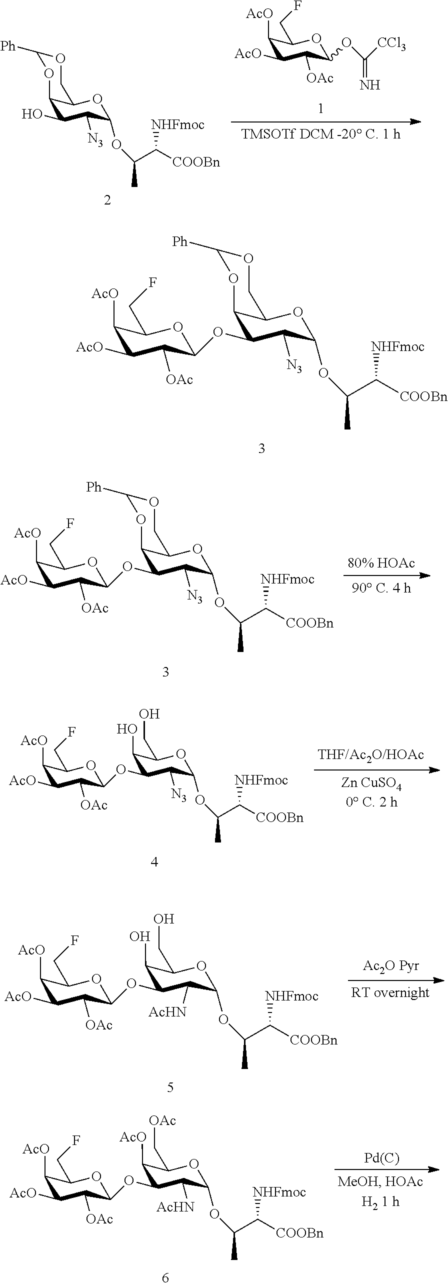

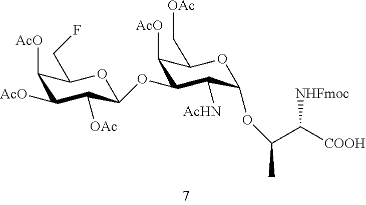

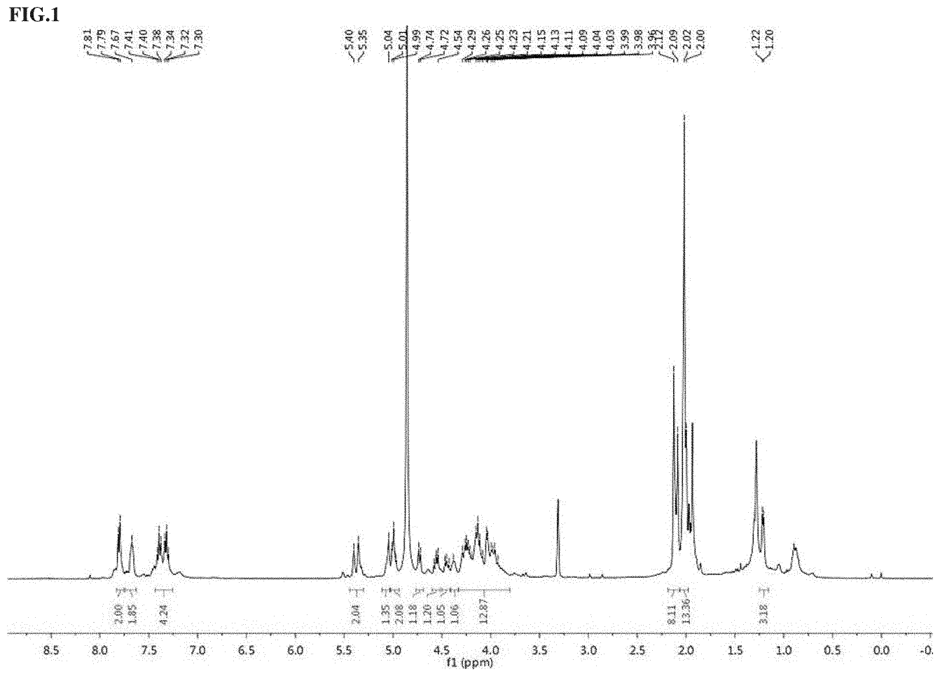

Firstly, 4 Å molecular sieve was added into a 50 mL branched flask and baked at 600° C. for 20 min, after cooling to room temperature, the fluoro-galactosyl donor 1 (410 mg, 905.55 μmol) and glycosyl receptor 2 (320 mg, 452.78 μmol) were added into the flask, 10 mL of anhydrous dichloromethane was added to the flask, and the system was placed at −20° C. after stirring at room temperature for 30 min, trimethylsilyl trifluoromethanesulfonate (TMSOTf, 35 μL, 181.11 μmol) was added dropwise and reacted at −20° C. for 1 h. After TLC (PE:EA=2:1, R_{f}=0.3) monitoring to the completion of the reaction, triethylamine quenching accelerator was added dropwise until the pH of the system was neutral. The molecular sieve was removed by suction filtration with a sand board funnel with diatomite, and anhydrous dichloromethane was removed by rotary evaporation, the silica gel column was used for separation and purification to obtain compound 3 (340 mg, 341.02 μmol, 75%). Then, compound 3 was dissolved in 80% glacial acetic acid aqueous solution, and the benzyl protecting group was removed by reaction in an oil bath at 90° C. to obtain compound 4. Subsequently, the azide group was reduced to N-acetyl group with zinc powder in a mixed solution of tetrahydrofuran/acetic anhydride/glacial acetic acid (3:2:1, v/v/v), and the exposed hydroxyl group of the compound was protected by acetylation under the condition of pyridine/acetic anhydride, the compound 6 was obtained. Finally, benzyl group was removed from threonine by hydrolysis in 10% palladium carbon and hydrogen gas streams in methanol as solvent. Compound 7 (126 mg, 137.12 μmol, as shown in FIG. 1) was obtained with a total yield of 31% through five steps. ¹H NMR (400 MHz, MeOD) δ 7.81 (d, J=7.4 Hz, 2H), 7.63 (dd, J=38.6, 6.1 Hz, 2H), 7.43-7.28 (m, 4H), 5.46-5.32 (m, 2H), 5.00 (d, J=6.2 Hz, 2H), 4.67 (d, J=6.8 Hz, 1H), 4.52 (m, 4H), 4.41-4.30 (m, 2H), 4.23 (m, 3H), 4.13 (dd, J=11.4, 4.5 Hz, 1H), 4.07-3.87 (m, 3H), 2.11 (d, J=9.2 Hz, 6H), 2.02 (d, J=10.0 Hz, 6H), 1.97 (s, 3H), 1.93 (s, 3H), 1.22 (d, J=6.3 Hz, 3H). ¹³C NMR (101 MHz, MeOD) δ 171.82, 170.93, 170.64, 170.10, 169.74, 143.85, 141.31, 127.48, 126.84, 124.69, 119.65, 101.00, 99.51, 81.49, 76.01, 73.28, 71.35, 71.12, 70.74, 69.79, 68.73, 67.46, 67.12, 66.22, 62.78, 58.46, 21.88, 19.58-18.96, 17.78. ¹⁹F NMR (376 MHz, CDCl₃) δ −232.54 (s).

(b) Solid Phase Synthesis of Fluoro-TF-MUC1 Glycopeptide 8

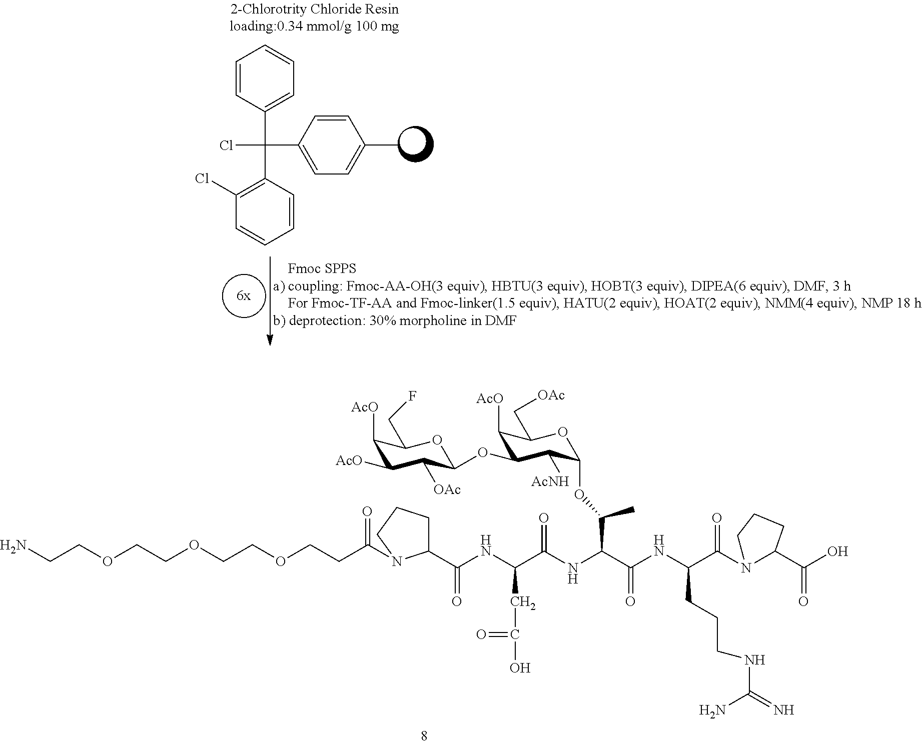

Fluoro-TF-MUC1 glycopeptide fragments was synthesized by Fmoc protected peptide solid phase synthesis method. The specific synthesis steps are as follows.

(i) Activation of resin: Firstly, 2-chlorotrityl chloride resin (0.34 mmol/g, 100 mg) was put into a 50 mL solid phase synthesis tube, and dried in vacuum at room temperature overnight, then 2 mL of anhydrous DCM was added into the solid-phase synthesis tube and placed on a shaking table (28° C., 240 r/min) and shaken for 30 min. Finally, the liquid was filtered off with suction.

(ii) Loading the first amino acid Fmoc-Pro-OH: the Fmoc-Pro-OH (35.4 mg, 0.11 mmol) was weighed and put in 2 mL of anhydrous DCM, it was added into the solid phase synthesis tube containing activated resin after dissolved by ultrasound, and then N, N-Diisopropyl ethylamine (DIEA, 52 μL) was added slowly into it. The solid phase synthesis tube was placed in a shaker (28° C., 240 r/min) and shaken for 3 h, methanol (15 μL) was added into the reaction mixture, and the reaction liquid was filtered out after being shaken for 30 min, the resin was washed alternately with 2 mL DMF/MeOH/DCM for 5 min each time. Finally, the resin was drained and dried in vacuum overnight.

(iii) Removing Fmoc: 30% morpholine was added into the resin, the reaction solution was drained after the shaker was shaken for 30 min, and then the operation was repeated once. After the reaction was complete, a small amount of resin was put into a test tube, ninhydrin/phenol chromogenic agent was added dropwise. The test tube was placed in an oil bath at 120° C., the color change of resin was observed after 5 min, and the resin turned blue overall when the reaction was complete. The resin was washed with DMF/isopropanol/DMF in sequence after the reaction was complete, and the resin was drained after washing.

(iv) Loading the second amino acid Fmoc-Arg(Pbf)-OH:Fmoc-Arg(Pbf)-OH (68.1 mg, 0.11 mmol), HBTU (39.8 mg, 0.11 mmol), HOBt (14.2 mg, 0.11 mmol) were weighed and added in 2 mL of DMF, and it was added into a solid phase synthesis tube containing resin after dissolution by ultrasonication, and then N,N-Diisopropyl ethylamine (DIEA, 35 μL) was added slowly to the mixture. The solid phase synthesis tube was placed on a shaking table (28° C., 240 r/min) and shaken for 3 h. After the reaction was complete, a small amount of resin was put into the test tube, it means the reaction is complete if all the resin turns white by observing the color change of resin. the resin was washed with DMF/isopropanol/DMF in sequence after the reaction was complete, and the resin was drained after washing. The operation of removing Fmoc was the same as (iii).

(v) Loading the third amino acid: fluoro-TF antigen (47 mg, 0.05 mmol) that previously synthesized, HATU (30 mg, 0.08 mmol), HOAt (9.3 mg, 0.068 mmol) were weighed and added in 2 mL of NMP, and it was added into a solid phase synthesis tube containing resin after dissolution by ultrasonication, and then N-Methylmorpholine (NMM, 15 μL) was added slowly to the mixture. The solid phase synthesis tube was placed on a shaking table (28° C., 240 r/min) and shaken for 18 h. After the reaction was complete, a small amount of resin was put into the test tube, it means the reaction is complete if all the resin turns white by observing the color change of resin. The resin was washed with DMF/isopropanol/DMF in sequence after the reaction was complete, and the resin was drained after washing. The operation of removing Fmoc was the same as (iii).

(vi) Loading the forth amino acid Fmoc-Asp(OtBu)-OH:Fmoc-Asp(OtBu)-OH (43.2 mg, 0.11 mmol), HBTU (39.8 mg, 0.11 mmol), HOBt (14.2 mg, 0.11 mmol) were weighed and added in 2 mL of DMF, and the subsequent operations were the same as (iv). The operation of removing Fmoc was the same as (iii).

(vii) Loading the fifth amino acid Fmoc-Pro-OH:Fmoc-Pro-OH (35.4 mg, 0.11 mmol), HBTU (39.8 mg, 0.11 mmol), HOBt (14.2 mg, 0.11 mmol) were weighed and added in 2 mL of DMF, and the subsequent operations were the same as (iv). The operation of removing Fmoc was the same as (iii).

(viii) Loading the Linker:Fmoc-NH-PEGS-CH₂CH₂COOH (45.2 mg, 0.102 mmol), HATU (38.8 mg, 0.102 mmol), HOAt (13.9 mg, 0.102 mmol) were weighed and added in 2 mL of NMP, and it was added into a solid phase synthesis tube containing resin after dissolution by ultrasonication, and then N-Methylmorpholine (23 μL, 0.2 umol) was added slowly to the mixture. The solid phase synthesis tube was placed on a shaking table (28° C., 240 r/min) and shaken for 3 h. After the reaction was complete, a small amount of resin was put into a test tube, it means the reaction is complete if all the resin turns white by observing the color change of resin. The resin was washed with DMF/isopropanol/DMF in sequence after the reaction was complete, and the resin was drained after washing. The operation of removing Fmoc was the same as (iii).

Cracking: 10 mL of the lysate (TFA:TIS:H₂O=15:0.9:0.9) was prepared, 5 mL of lysate was added to the resin, the reaction liquid was filtered into the cooled ether for recrystallization after being shaken for 2 h on a shaker (34° C., 240 r/min), then the remaining 5 mL of lysate was added into the resin again for reaction for 2 h, and the filtrate was combined in the cooled ether, and then it was left standing at −20° C. for 2 h. Finally, the white solid precipitated from the ether was collected in a centrifuge tube by centrifugation, and the glycopeptide compound 8 (15 mg, 10.6 μmol, 31%) was obtained by argon drying.

(c) Synthesis of Fluoro-TF-MUC1 Glycopeptide Conjugate

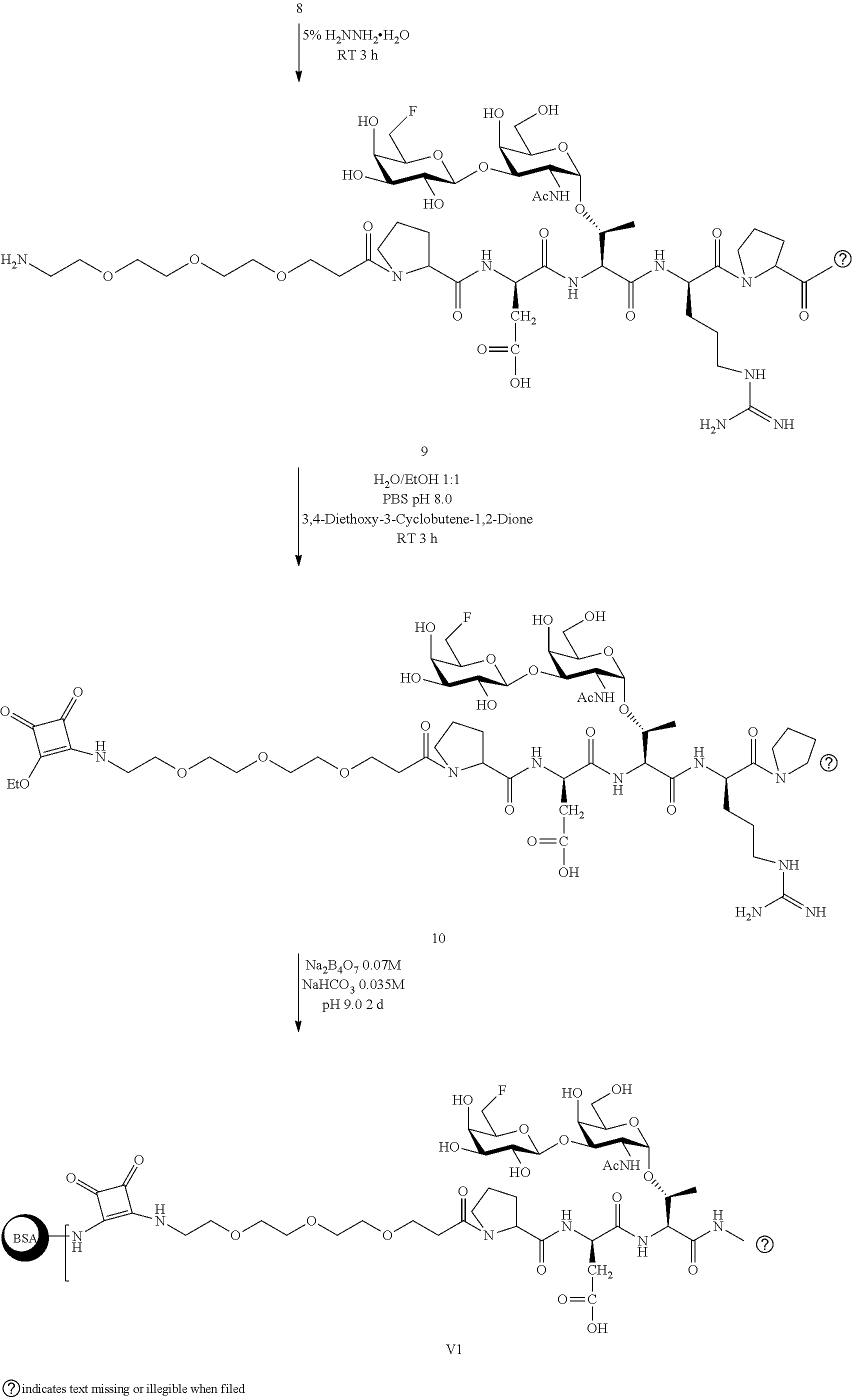

Glycopeptide compound 8 (15 mg, 10.6 μmol) was put into a flask of 10 mL, 2 mL of hydrazine hydrate was added in the flask, and it was stirred at room temperature for 3 h, rotary evaporation and concentration were conducted after the reaction was complete. The molecular weight of glycopeptide compound 8 (15 mg, 10.6 μmol) was determined by MALDI-TOF MS. Deacetylation product 9 (10 mg, 8.7 μmol, 82%) was purified on C18 reversed-phase semi-preparative liquid column.

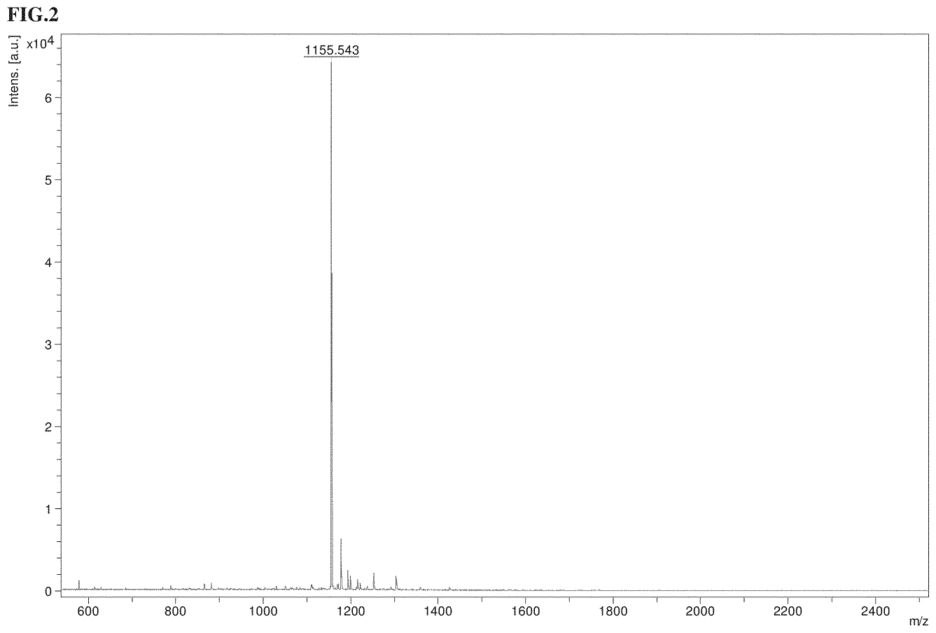

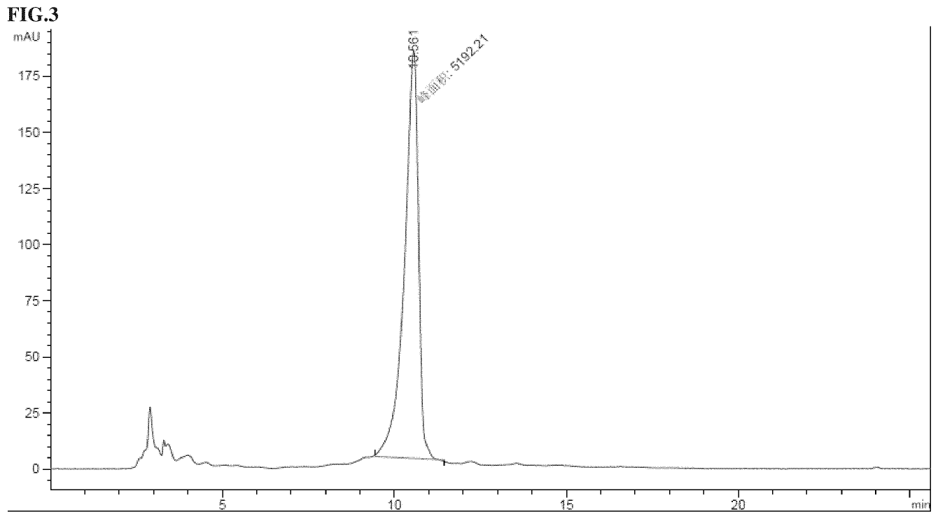

Compound 9 (5 mg, 4.3 μmol) was dissolved in EtOH/H₂O (2 mL, 1:1), then diethyl squarate (3.84 μL, 26.1 μmol) was slowly added, and saturated Na₂CO3 solution was added dropwise to the reaction solution (pH 8.0). After stirring at room temperature for 3 h, glacial acetic acid was added dropwise to quench the reaction. The reaction solution was concentrated by rotary evaporation, and its molecular weight was determined by MALDI-TOF MS using DHB as a matrix. Purified by C18 reversed-phase semi-preparative liquid column, product 10 (4.9 mg, 3.8 μmol, 88%) was obtained by lyophilized. Characterization of molecular weight and purity of compound 10: MALDI-TOF MS: m/z for C₄₇H₇₉FN₁₀O₂₂ [M+H]⁺ calcd: 1155.543, found: 1155.543, as shown in FIG. 2. C18 analytical HPLC: mobile phase A: acetonitrile (containing 0.1% TFA); mobile phase B: water; retention time Rt=10.5 min (gradient elution 30 min, 5-30% mobile phase A, C-18 column, k=220 nm), as shown in FIG. 3.

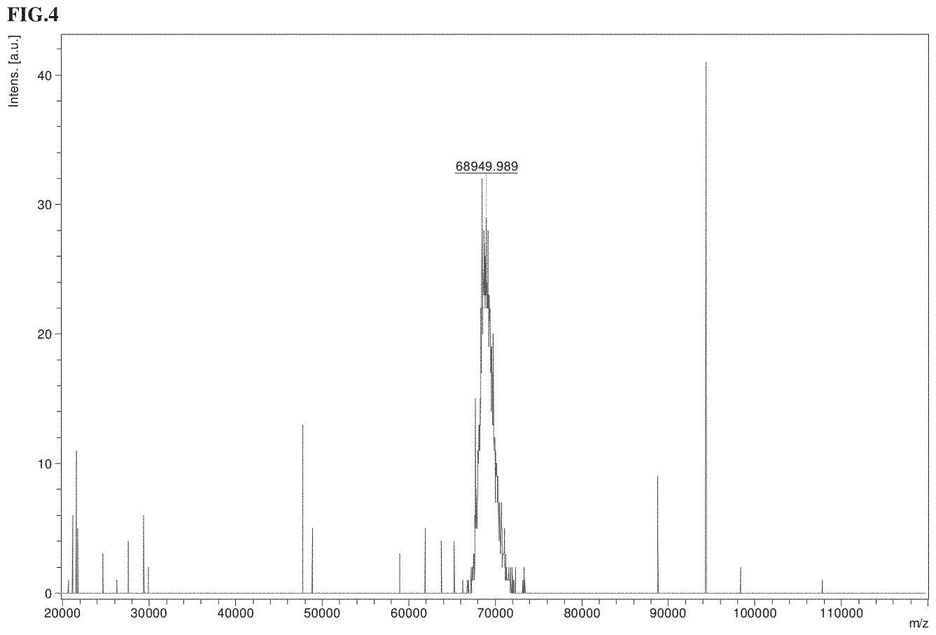

Protein BSA (3.2 mg, 0.048 μmol) was dissolved in 0.5 mL of buffer (Na₂B₄O₇ 0.07 mol/L, KHCO₃ 0.035 mol/L, pH9.0), and compound 10 (3 mg, 2.3 μmol) was added. The mixture was placed on a shaker and shaken slowly at room temperature for 2 days. After the reaction, a white solid was obtained by ultrafiltration and lyophilization, namely fluoro-TF-MUC1 glycopeptide conjugate V1 (fluoro-TF-MUC1-BSA, 2.9 mg). The binding amount of glycopeptide and protein was determined by MALDI-TOF MS, as shown in FIG. 4.

(d) Immunogenicity Determination of Fluoro-TF-MUC1 Glycopeptide Conjugate

Balb/c mice of 6-8 weeks old were selected, with four mice in each group, the mice in the experimental group were subcutaneously injected in the neck with the fluoro-TF-MUC1 glycopeptide conjugate V1 prepared in Step 3 and the control drug V2 (TF-MUC1-BSA) solution by the same method respectively, each mouse was injected with 1 μg glycopeptide (50 μLPBS+50 μL emulsion of Freund's adjuvant) each time, immunized three times, the interval between each immunization was two weeks, complete Freund's adjuvant was used for the first immunization, and incomplete Freund's adjuvant was used in the later two immunizations. Blood samples were collected from the eyeballs of mice in the experimental group and the control group two weeks after the third immunization (the 42nd day), and then the blood was centrifuged twice at 2500 rpm/min for 10 minutes each time, the upper serum was collected and stored in the EP tube at −20° C.

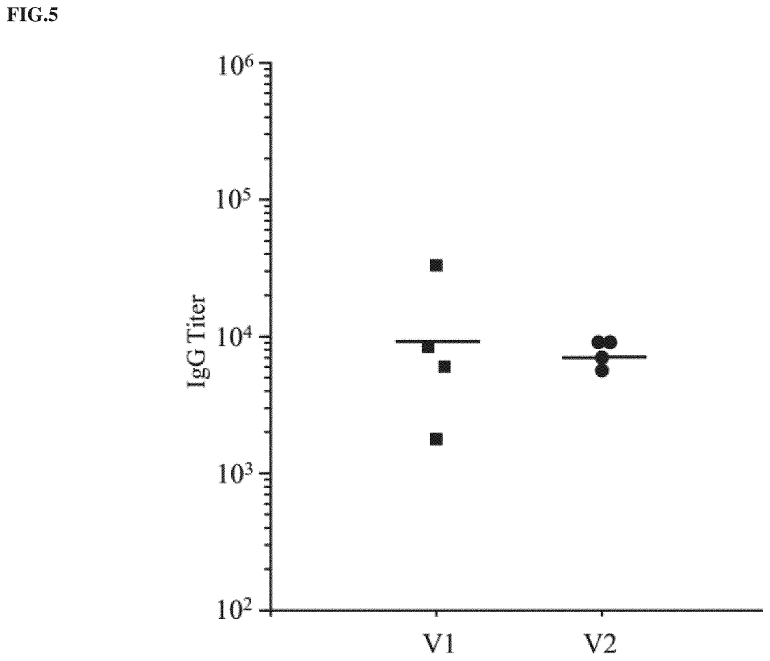

An antiserum was prepared to study its immunogenicity. The OVA conjugate of the glycopeptide of the reference substance was used as the fixed antigen, and the titer of the glycopeptide-specific antibody was detected by enzyme-linked immunoassay (ELISA). The results of the immunotiter are shown in FIG. 5. After immunization, the titer of IgG antibody in the serum of mice was significantly increased, and the immune response was strong. It shows that the immune response induced by conjugate is mainly IgG type, which belongs to the immune response participated by T cells, and the response enables the host cell to produce immune memory and promote antibody maturation. The results of animal immunity experiments show that the fluoro-TF-MUC1 glycopeptide conjugate is a very potential tumor vaccine.

Although the embodiments of the present application have been disclosed for illustrative purposes, those skilled in the art may understand that: various substitutions, variations, and modifications are possible within the spirit and scope of the present application and the claims attached thereto, and therefore, the scope of the present application is not limited to the contents disclosed in the embodiments.

1. A fluoro-TF-MUC1 glycopeptide conjugate, wherein the structural formula of the glycopeptide conjugate is as follows: 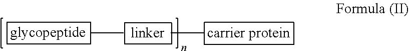
wherein, the glycopeptide is any glycopeptide in the following general formula (I): 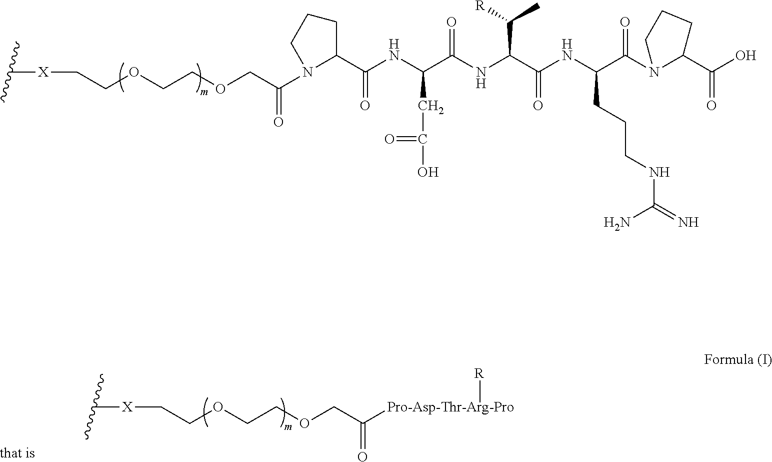
in formula (I) and formula (II):
m is an integer selected from 0 to 30;
R is selected from GalNAcα, GalNAcβ, GlcNAcα, GlcNAcβ, Galβ1-3GalNAcα, Galβ1-3GalNAcβ and fluoro derivatives of the glycogroups thereof;
X is selected from —CH₂, —NH—, —O—, —C(O)—, —S—, and 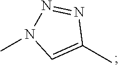
the linker is selected from a structural part obtained by directly or indirectly connecting the glycopeptide and the carrier protein;
n is the number of oligosaccharides linked to the carrier protein, and n is an integer selected from 0 to 30;
the carrier protein is selected from: bovine serum albumin, human serum albumin, hemocyanin, tetanus toxin, diphtheria toxin and non-toxic mutant of diphtheria toxin.

2. The fluoro-TF-MUC1 glycopeptide conjugate according to claim 1, wherein the general structural formula of the glycopeptide conjugate is selected from one of the follows: 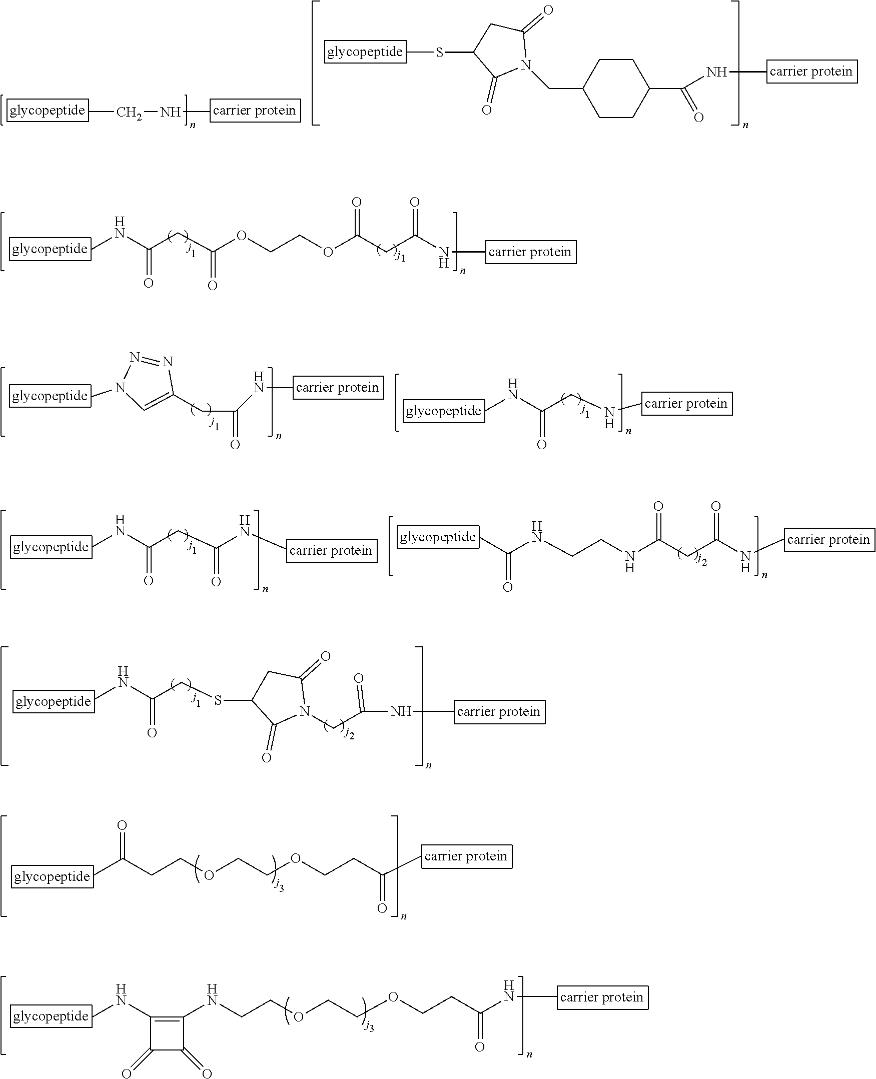
in the formula, j₁ is an integer selected from 0 to 10, j₂ is an integer selected from 0 to 10, j₃ is an integer selected from 0 to 10, n is an integer selected from 0 to 30.

3. The fluoro-TF-MUC1 glycopeptide conjugate according to claim 1, wherein the general structural formula of the glycopeptide conjugate is as follows: 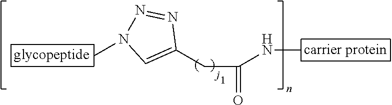
in the formula, j₁ is an integer selected from 0 to 10, n is an integer selected from 0 to 30.

4. The fluoro-TF-MUC1 glycopeptide conjugate according to claim 1, wherein the general structural formula of the glycopeptide conjugate is as follows: 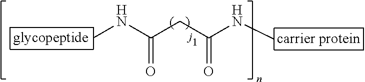
in the formula, j₁ is an integer selected from 0 to 10, n is an integer selected from 0 to 30.

5. The fluoro-TF-MUC1 glycopeptide conjugate according to claim 1, wherein the general structural formula of the glycopeptide conjugate is as follows: 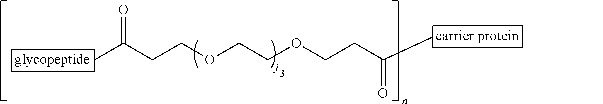
in the formula, j₃ is an integer selected from 0 to 10, n is an integer selected from 0 to 30.

6. The fluoro-TF-MUC1 glycopeptide conjugate according to claim 1, wherein the general structural formula of the glycopeptide conjugate is as follows: 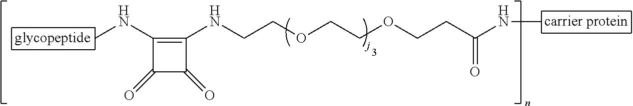
in the formula, j₃ is an integer selected from 0 to 10, n is an integer selected from 0 to 30.

7. A method for preparing fluoro-TF-MUC1 glycopeptide conjugate according to claim 1, wherein including the following technical routes:

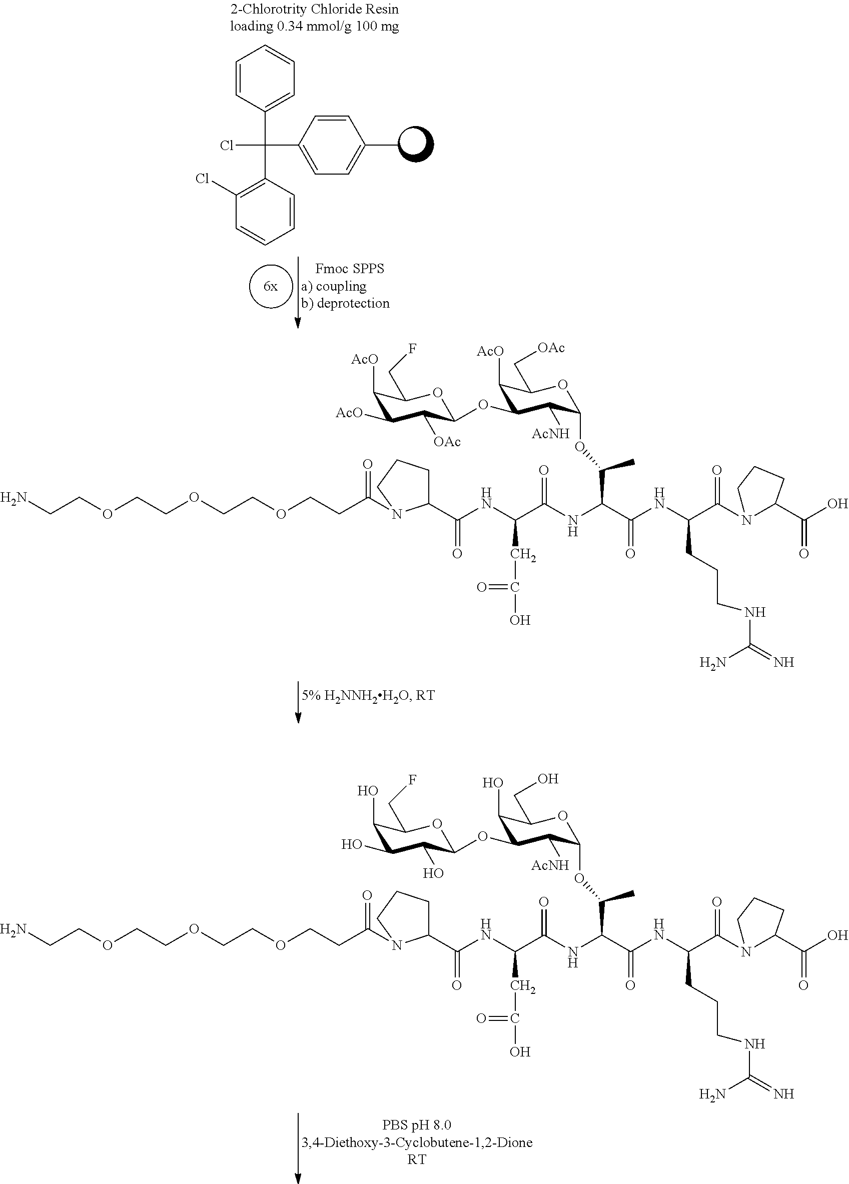

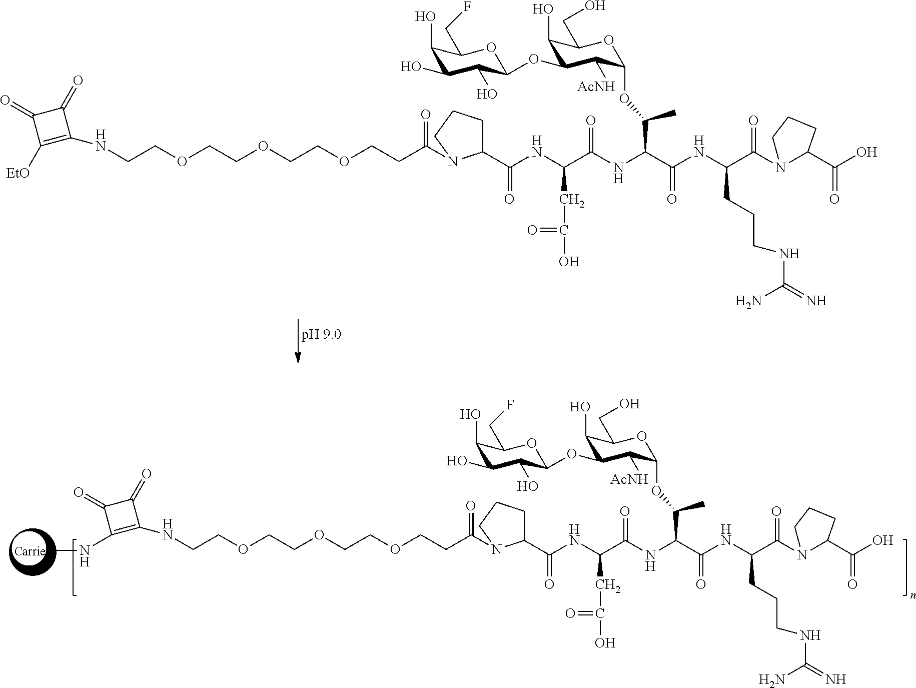

8. A use of the fluoro-TF-MUC1 glycopeptide conjugate according to claim 1 in the preparation of vaccines.

9. The use according to claim 8, wherein the vaccine is a tumor vaccine.

10. The use according to claim 8, wherein the general structural formula of the glycopeptide conjugate is selected from one of the follows: 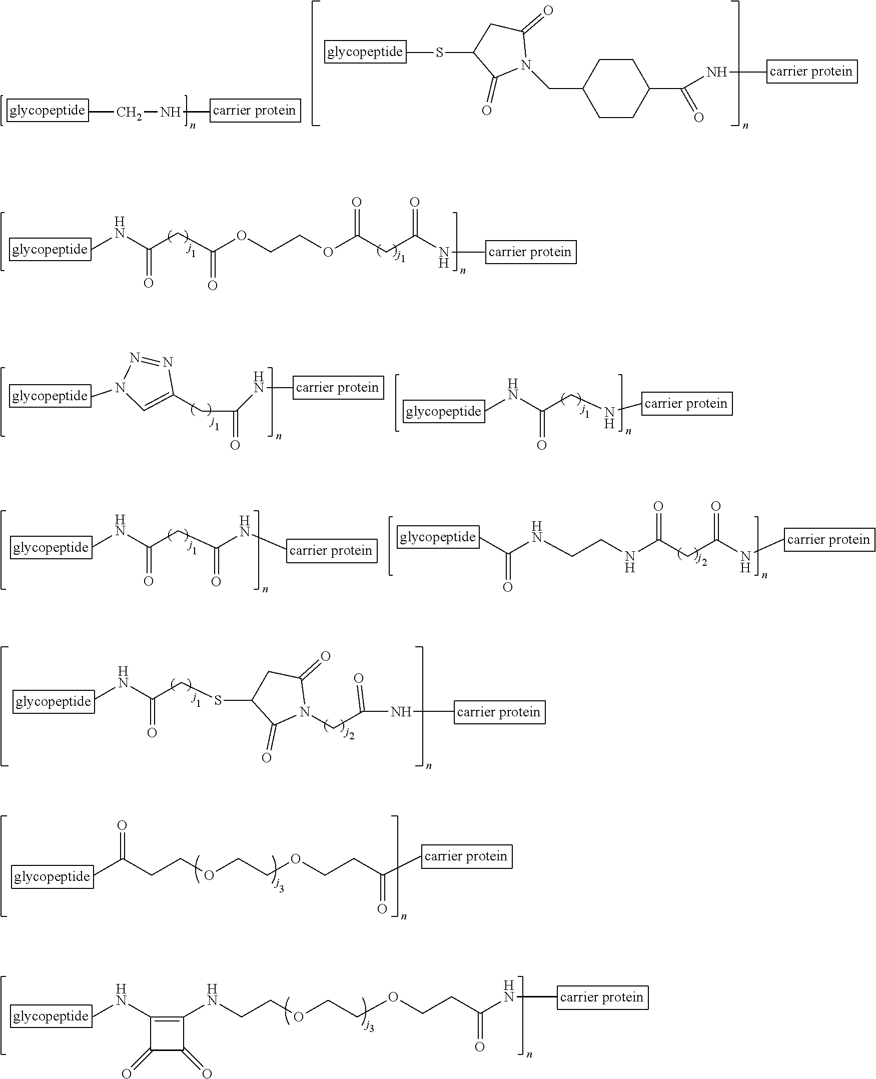
in the formula, j₁ is an integer selected from 0 to 10, j₂ is an integer selected from 0 to 10, j₃ is an integer selected from 0 to 10, n is an integer selected from 0 to 30.

11. The use according to claim 10, wherein the vaccine is a tumor vaccine.

12. The use according to claim 8, wherein the general structural formula of the glycopeptide conjugate is as follows: 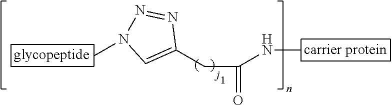
in the formula, j₁ is an integer selected from 0 to 10, n is an integer selected from 0 to 30.

13. The use according to claim 12, wherein the vaccine is a tumor vaccine.

14. The use according to claim 8, wherein the general structural formula of the glycopeptide conjugate is as follows: 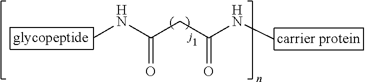
in the formula, j₁ is an integer selected from 0 to 10, n is an integer selected from 0 to 30.

15. The use according to claim 14, wherein the vaccine is a tumor vaccine.

16. The use according to claim 8, wherein the general structural formula of the glycopeptide conjugate is as follows: 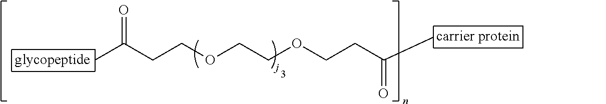
in the formula, j₃ is an integer selected from 0 to 10, n is an integer selected from 0 to 30.

17. The use according to claim 16, wherein the vaccine is a tumor vaccine.

18. The use according to claim 8, wherein the general structural formula of the glycopeptide conjugate is as follows: 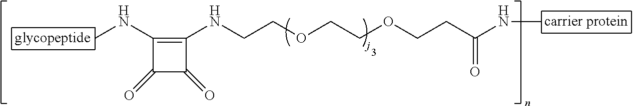
in the formula, j₃ is an integer selected from 0 to 10, n is an integer selected from 0 to 30.

19. The use according to claim 18, wherein the vaccine is a tumor vaccine.


SEQUENCE LISTING (1 sequence)

SEQ ID NO: 1 - protein, 20 aa, Unknown
  repeat sequence of MUC1
HGVTSAPDTRPAPGSTAPPA